(19) United States
(12) Patent Application Publication
(10) Pub. No.: US 2014/0180090 A1
(43) Pub. Date: Jun. 26, 2014

(54) ULTRASOUND DIAGNOSTIC APPARATUS, TISSUE ELASTICITY MEASUREMENT METHOD, AND RECORDING MEDIUM

(71) Applicant: FUJIFILM Corporation, Tokyo (JP)

(72) Inventors: Yasunori OHTA, Ashigara-kami-gun (JP); Tsuyoshi TANABE, Ashigara-kami-gun (JP); Noriaki IDA, Ashigara-kami-gun (JP); Shin NAKATA, Ashigara-kami-gun (JP)

(73) Assignee: FUJIFILM Corporation, Tokyo (JP)

(21) Appl. No.: 14/140,091

(22) Filed: Dec. 24, 2013

(30) Foreign Application Priority Data
Dec. 25, 2012 (JP) 2012-281024

(51) Int. Cl. A61B 8/08

(52) U.S. Cl. CPC A61B 8/485
USPC 600/438

(57) ABSTRACT

When tissue elasticity of a subject is measured, an ultrasound diagnostic apparatus sets a sound velocity for each of segment regions established by dividing the subject, processes reception signals output by a piezoelectric element array based on the set sound velocities, and performs tissue elasticity measurement based on the reception signals processed based on the set sound velocities. Owing to this configuration, when tissue elasticity of a subject is measured, the ultrasound diagnostic apparatus prevents the accuracy of tissue elasticity measurement from deteriorating due to image distortions.

### BACKGROUND OF THE INVENTION

The present invention relates to an ultrasound diagnostic apparatus, particularly to an ultrasound diagnostic apparatus, a tissue elasticity measurement method, and a recording medium for measuring tissue elasticity of a subject.

Ultrasound diagnostic apparatuses using ultrasound images are put to practical use in the medical field.

In general, this type of ultrasound diagnostic apparatus includes an ultrasound probe (hereinafter also called “probe”) having a piezoelectric element array in which piezoelectric elements transmitting and receiving ultrasonic waves are arranged, and a diagnostic apparatus body.

The ultrasound diagnostic apparatus transmits ultrasonic waves from the probe into a subject's body, receives the ultrasonic echo from the subject with the probe, and electrically processes the resulting reception signals with the diagnostic apparatus body to produce an ultrasound image.

The piezoelectric element array of the ultrasound probe receives through a plurality of piezoelectric elements an ultrasonic echo resulted from one transmission of an ultrasonic beam. Accordingly, even though an ultrasonic echo results from reflection at the same reflection point, the time taken to enter each piezoelectric element varies depending on the position of the piezoelectric element.

To cope with it, the ultrasound diagnostic apparatus performs delay correction on reception signals output from the ultrasound probe for the respective piezoelectric elements using a delay time corresponding to, for example, the position of each piezoelectric element, followed by addition (matching addition), thereby producing a proper ultrasound image without distortion.

Aside from that, in recent years, tissue elasticity measurement (elastography) of a living body for measuring the elasticity of a body tissue by an ultrasound diagnostic apparatus is put into practical use.

While stiff tissues in a living body hardly exhibit strain by pressure, tissues applied with pressure exhibit higher strain as they are softer. In the tissue elasticity measurement by the ultrasound diagnostic apparatus, for instance, an ultrasound probe is used to press the subject, and the correlation between ultrasound images produced before and after applying pressure or between two ultrasound images produced with a different degree of pressure (the displacement in the same region of the subject caused by applied pressure) is utilized to measure the elasticity of the inside of the tissue (JP 2001-519674 A, JP 2010-69006 A and JP 4221555 B).

### SUMMARY OF THE INVENTION

In such tissue elasticity measurement by the ultrasound diagnostic apparatus, ultrasound images produced before and after applying pressure or ultrasound images produced with a different degree of pressure may be distorted. When one of ultrasound images is distorted in the tissue elasticity measurement, the result of the tissue elasticity measurement is affected by the distortion and it hinders an accurate diagnosis.

An object of the present invention is to solve the foregoing drawback in the prior art and, as the first aspect, to provide an ultrasound diagnostic apparatus which enables to accurately measure tissue elasticity of a subject.

An object of the present invention is, as the second aspect, to provide a tissue elasticity measurement method which enables to accurately measure tissue elasticity of a subject.

An object of the present invention is, as the third aspect, to provide a recording medium which enables to accurately measure tissue elasticity of a subject.

In order to attain the object, the present invention provides an ultrasound diagnostic apparatus comprising:

a piezoelectric element array having piezoelectric elements arranged therein, each adapted to transmit ultrasonic waves, receive an ultrasonic echo reflected by a subject, and output a reception signal according to received ultrasonic waves;

a controller adapted to control transmission and reception of ultrasonic waves by the piezoelectric element array;

a storage unit adapted to store the reception signal output by the piezoelectric element array;

a sound velocity setting unit adapted to divide the subject into multiple segment regions and set a sound velocity for each of the segment regions with use of the reception signal stored in the storage unit;

an image producer adapted to produce an ultrasound image by processing the reception signal output by the piezoelectric element array or the reception signal read out from the storage unit based on the sound velocity for each of the segment regions;

a tissue elasticity calculator adapted to calculate tissue elasticity of the subject with use of the reception signal processed by the image producer; and

an image display unit,

wherein when the tissue elasticity calculator calculates the tissue elasticity,

the controller causes the piezoelectric element array to perform transmission and reception of ultrasonic waves for sound velocity setting so that the sound velocity setting unit sets a sound velocity,

the sound velocity setting unit sets the sound velocity for each of the segment regions of the subject with use of the reception signal acquired through the transmission and reception of ultrasonic waves for sound velocity setting, and

the tissue elasticity calculator calculates the tissue elasticity of the subject with use of the reception signal processed by the image producer based on the sound velocity set through the transmission and reception of ultrasonic waves for sound velocity setting.

Preferably, in the ultrasound diagnostic apparatus according to the present invention, the image producer produces the ultrasound image by processing the reception signal output by the piezoelectric element array based on the sound velocity set through the transmission and reception of ultrasonic waves for sound velocity setting when the tissue elasticity calculator calculates the tissue elasticity.

Preferably, a calculation result of the tissue elasticity calculated by the tissue elasticity calculator and the ultrasound image produced by the image producer are displayed on the image display unit.

Preferably, the image display unit performs display in at least one of an arrangement having an image in which the calculation result of the tissue elasticity overlaps the ultrasound image; an arrangement having the calculation result of the tissue elasticity and the ultrasound image arranged side by side; an arrangement having the image in which the calculation result of the tissue elasticity overlaps the ultrasound image and the ultrasound image arranged side by side; and an arrangement having the image in which the calculation result of the tissue elasticity overlaps the ultrasound image and the calculation result of the tissue elasticity arranged side by side.

Preferably, the ultrasound diagnostic apparatus further comprises a displacement detector adapted to detect displacement of the piezoelectric element array,

wherein when the tissue elasticity calculator calculates the tissue elasticity of the subject, in a case where the displacement detector detects that there is no displacement of the piezoelectric element array, the controller does not allow the piezoelectric element array to perform the transmission and reception of ultrasonic waves for sound velocity setting, and the tissue elasticity calculator and the image producer process the reception signal based on a sound velocity of the subject most recently set by the sound velocity setting unit.

Preferably, the displacement detector is at least one of a pressure gauge provided at an ultrasound probe of the ultrasound diagnostic apparatus, a displacement sensor adapted to detect displacement of the piezoelectric element array, and an image analyzer adapted to analyze the reception signal processed by the image producer.

Preferably, the tissue elasticity calculator calculates the tissue elasticity of the subject with use of the reception signal acquired through the transmission and reception of ultrasonic waves for sound velocity setting or with use of the reception signal acquired through subsequent transmission and reception of ultrasonic waves as carried out immediately after the transmission and reception of ultrasonic waves for sound velocity setting.

Preferably, the image producer produces the ultrasound image with use of the reception signal acquired through the transmission and reception of ultrasonic waves for sound velocity setting or with use of the reception signal acquired through subsequent transmission and reception of ultrasonic waves as carried out immediately after the transmission and reception of ultrasonic waves for sound velocity setting when the tissue elasticity calculator calculates the tissue elasticity of the subject.

The present invention provides a tissue elasticity measurement method, comprising the steps of:

at a time when tissue elasticity of a subject is measured by an ultrasound diagnostic apparatus,

causing a piezoelectric element array having piezoelectric elements arranged therein, each adapted to transmit ultrasonic waves, receive an ultrasonic echo reflected by the subject, and output a reception signal according to received ultrasonic waves, to perform transmission and reception of ultrasonic waves for sound velocity setting for setting a sound velocity of the subject;

setting the sound velocity for each of segment regions established by dividing the subject based on the reception signal acquired through the transmission and reception of ultrasonic waves for sound velocity setting; and

calculating the tissue elasticity of the subject with use of the reception signal which has been output from the piezoelectric element array and processed based on the sound velocity set through the transmission and reception of ultrasonic waves for sound velocity setting.

Preferably, the tissue elasticity measurement method according to the present invention further comprises the step of:

producing an ultrasound image by processing the reception signal output by the piezoelectric element array based on the sound velocity set through the transmission and reception of ultrasonic waves for sound velocity setting.

The present invention provides a recording medium having stored therein a program that causes a computer to implement:

a step of causing a piezoelectric element array having piezoelectric elements arranged therein, each adapted to transmit ultrasonic waves, receive an ultrasonic echo reflected by a subject, and output a reception signal according to received ultrasonic waves, to perform transmission and reception of ultrasonic waves for sound velocity setting for setting a sound velocity of the subject;

a step of setting the sound velocity for each of segment regions established by dividing the subject based on the reception signal acquired through the transmission and reception of ultrasonic waves for sound velocity setting; and

a step of calculating the tissue elasticity of the subject with use of the reception signal which has been output from the piezoelectric element array and processed based on the sound velocity set through the transmission and reception of ultrasonic waves for sound velocity setting.

Preferably, the stored program causes the computer to further implement a step of producing an ultrasound image by processing the reception signal output by the piezoelectric element array based on the sound velocity set through the transmission and reception of ultrasonic waves for sound velocity setting.

According to the present invention, an ultrasound image (sound ray signal) used in tissue elasticity measurement can be produced based on an appropriate sound velocity in a subject applied with pressure. Specifically, all ultrasound images used in tissue elasticity measurement are subjected to delay correction based on an appropriate sound velocity. Preferably, a high-quality ultrasound image produced during the tissue elasticity measurement based on an appropriate sound velocity in a subject applied with pressure is displayed along with a result of the tissue elasticity measurement.

Hence, according to the present invention, an accurate diagnosis can be performed with the use of the accurate result of the tissue elasticity measurement and possibly the high-quality ultrasound image.

### BRIEF DESCRIPTION OF THE DRAWINGS

FIG. 1 is a block diagram conceptually showing an ultrasound diagnostic apparatus of the invention.

FIG. 2 is a conceptual diagram for explaining tissue elasticity measurement in the ultrasound diagnostic apparatus shown in FIG. 1.

### DETAILED DESCRIPTION OF THE INVENTION

An ultrasound diagnostic apparatus, a tissue elasticity measurement method, and a recording medium of the invention will be described below in detail with reference to preferred embodiments shown in the accompanying drawings.

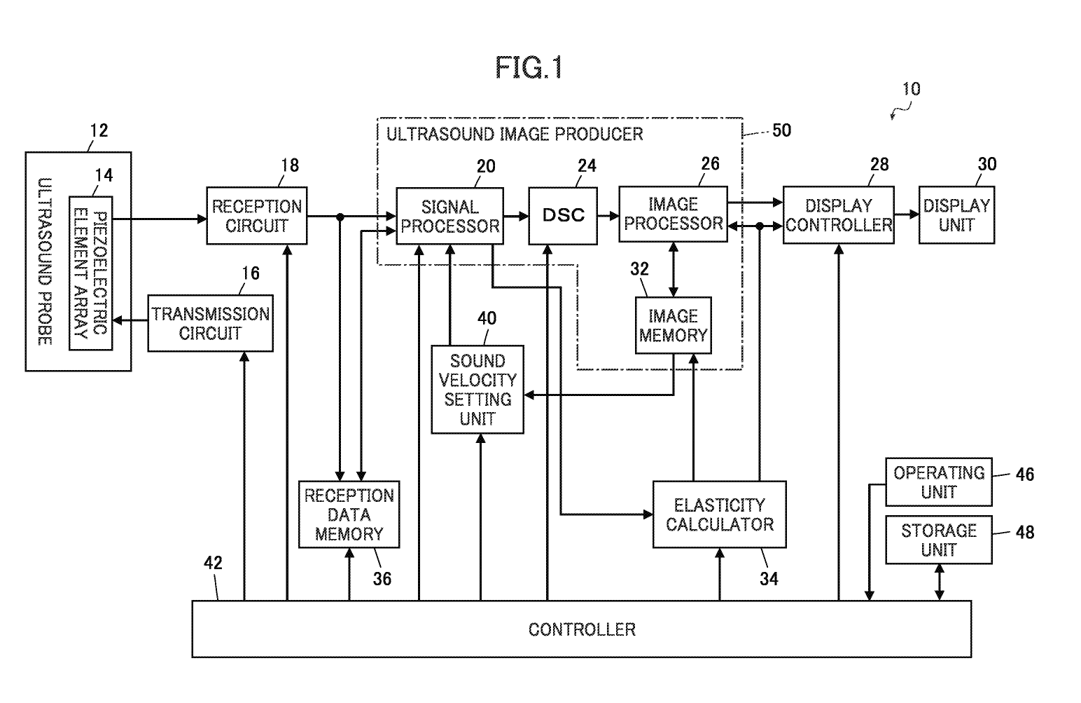

FIG. 1 is a block diagram conceptually showing an example of an ultrasound diagnostic apparatus of the invention which implements a tissue elasticity measurement method of the invention.

As shown in FIG. 1, an ultrasound diagnostic apparatus 10 has an ultrasound probe 12 (hereinafter called “probe 12”) including a piezoelectric element array 14.

The piezoelectric element array 14 of the probe 12 is connected to a transmission circuit 16 and a reception circuit 18. The reception circuit 18 is connected in sequence to a signal processor 20, a digital scan converter (DSC) 24, an image processor 26, a display controller 28, and a display unit 30. The image processor 26 is connected to an image memory 32.

The signal processor 20, the DSC 24, the image processor 26, and the image memory 32 constitute an ultrasound image producer 50.

The signal processor 20, the image processor 26, the display controller 28, and the image memory 32 are connected to an elasticity calculator 34.

The reception circuit 18 and the signal processor 20 are connected to a reception data memory 36, and the image memory 32 and the signal processor 20 are connected to a sound velocity setting unit 40.

Furthermore, the transmission circuit 16, the reception circuit 18, the signal processor 20, the DSC 24, the display controller 28, the elasticity calculator 34, the reception data memory 36, and the sound velocity setting unit 40 are connected to a controller 42. The controller 42 is also connected to an operating unit 46 and a storage unit 48.

In the illustrated example, the transmission circuit 16, the reception circuit 18, the ultrasound image producer 50, the display controller 28, the display unit 30, the elasticity calculator 34, the reception data memory 36, the sound velocity setting unit 40, the controller 42, the operating unit 46, and the storage unit 48 constitute a diagnostic apparatus body of the ultrasound diagnostic apparatus 10.

The diagnostic apparatus body is configured by using, for example, a computer.

The piezoelectric element array 14 of the probe 12 includes a plurality of piezoelectric elements (ultrasound transducers) arranged one-dimensionally or two-dimensionally. These piezoelectric elements each transmit ultrasonic waves according to driving signals supplied from the transmission circuit 16 and receive ultrasonic echoes from the subject to output reception signals.

The piezoelectric element is composed of a vibrator in which electrodes are provided at the both ends of a piezoelectric body. The piezoelectric body may be composed of, for example, a piezoelectric ceramic typified by lead zirconate titanate (PZT), a piezoelectric polymer typified by polyvinylidene fluoride (PVDF), or a piezoelectric monocrystal typified by lead magnesium niobate-lead titanate solid solution (PMN-PT).

When a pulsed voltage or a continuous-wave voltage is applied to the electrodes of such a vibrator, the piezoelectric body expands and contracts to cause the vibrator to generate pulsed or continuous ultrasonic waves. The ultrasonic waves generated at the respective vibrators are synthesized to form an ultrasonic beam.

Further, upon reception of propagating ultrasonic waves, the vibrators expand and contract to produce electric signals. The electric signals are output from the piezoelectric elements (piezoelectric element array 14) as reception signals of the ultrasonic waves.

The transmission circuit 16 includes, for instance, a plurality of pulse generators. The transmission circuit 16 adjusts delay amounts of the driving signals and then supplies the adjusted driving signals to the respective piezoelectric elements so that the ultrasonic waves transmitted from the piezoelectric element array 14 form an ultrasonic beam as desired. The transmission circuit 16 adjusts each delay amount based on a transmission delay pattern selected in accordance with a control signal from the controller 42.

The reception circuit 18 amplifies the reception signals transmitted from the piezoelectric elements of the piezoelectric element array 14 and analog-to-digital converts the amplified signals to produce pieces of digitalized reception data as many as the number of reception channels.

Based on sound velocities (a set sound velocity and an optimal sound velocity to be described later) input from the sound velocity setting unit 40, the signal processor 20 performs dedicated delay correction on each of the pieces of reception data produced by the reception circuit 18 to produce pieces of delay correction data. Further, the signal processor 20 adds those pieces of delay correction data (performs matching addition) to perform a reception focusing process. By this process, the ultrasonic echo is well focused so as to produce a sound ray signal (sound ray data).

Furthermore, the signal processor 20 corrects the sound ray signals for the attenuation due to distance according to the depth at which the ultrasonic waves are reflected, and then performs an envelope detection process. By this process, the signal processor 20 produces a B-mode image signal (ultrasound image) which is tomographic image information relating to the tissue in the subject.

The DSC 24 converts the B-mode image signal produced by the signal processor 20 into an image signal compatible with an ordinary television signal scanning mode (raster conversion).

The image processor 26 performs various kinds of necessary image processing such as gradation processing on the B-mode image signal entered from the DSC 24, and outputs the B-mode image signal to the display controller 28. Alternatively, the image processor 26 stores the B-mode image signal having been subjected to the necessary processing in the image memory 32.

As described above, the ultrasound image producer 50 is made up of the signal processor 20, the DSC 24, the image processor 26, and the image memory 32.

Based on the B-mode image signal having been subjected to the image processing by the image processor 26, various kinds of information input by the operating unit 46, a result of elasticity measurement by the elasticity calculator 34, and the like, the display controller 28 causes the display unit 30 to display an ultrasound diagnostic image, the elasticity measurement result and the like.

The display unit 30 includes a display device such as an LCD, for example, and displays the ultrasound diagnostic image under the control of the display controller 28. In this example, the display controller 28 and the display unit 30 are capable of displaying color images.

When performing the elasticity measurement of the subject, the elasticity calculator 34 acquires the cross-correlation between corresponding sound ray signals produced by the signal processor 20 with respect to two ultrasound images produced before and after pressing the subject or two ultrasound images produced with a different degree of pressure, and calculates tissue elasticity of the subject.

It should be noted that, in the present invention, as a method of tissue elasticity calculation performed by the elasticity calculator 34, known methods in which ultrasound images produced before and after pressing the subject or ultrasound images produced with a different degree of pressure are used to acquire the correlation between the images, thereby calculating tissue elasticity of the subject, are all applicable.

The reception data memory 36 sequentially stores the reception data output from the reception circuit 18 and stores the delay correction data produced by the signal processor 20.

The sound velocity setting unit 40 sets optimal sound velocities that are sound velocities of (the inside of) the subject.

In the present invention, the sound velocity setting unit 40 divides the inside of the subject into multiple segment regions and sets an optimal sound velocity for each of the segment regions. As an example, the sound velocity setting unit 40 provides a predetermined set sound velocity to the signal processor 20 and causes the ultrasound image producer 50 to produce B-mode image signals while changing the set sound velocity. The sound velocity setting unit 40 analyzes B-mode images thus produced with the different set sound velocities and sets a sound velocity at which the contrast or the sharpness of the image is highest as the optimal sound velocity of each segment region of the subject.

The controller 42 controls components of the ultrasound diagnostic apparatus according to instructions entered by the operator using the operating unit 46.

The operating unit 46 is provided for the operator to perform input operations and may be composed of, for example, a keyboard, a mouse, a track ball, and/or a touch panel.

The storage unit 48 stores, for example, an operation program and may be constituted by, for example, a recording medium such as a hard disk, a flexible disk, an MO, an MT, a RAM, a CD-ROM, a DVD-ROM, an SD card, a CF card, and a USB memory, or a server.

The signal processor 20, the DSC 24, the image processor 26, the display controller 28, and the sound velocity setting unit 40 are each constituted by a CPU and an operation program for causing the CPU to perform various kinds of processing, but they may be each constituted by a digital circuit.

The present invention will be explained in further detail by explaining the operation of the tissue elasticity measurement of the subject by the ultrasound diagnostic apparatus 10. A recording medium according to the present invention is a recording medium that has a program recorded therein for causing a computer to implement the tissue elasticity measurement method of the invention to be described below and is readable by a computer.

An elasticity measurement mode used when measuring tissue elasticity of the subject as set in the ultrasound diagnostic apparatus 10. In response to selection of the elasticity measurement mode through the operation of the operating unit 46, the ultrasound diagnostic apparatus 10 measures tissue elasticity of the subject.

Upon selection of the elasticity measurement mode, the controller 42 provides instructions to the transmission circuit 16 and the reception circuit 18 so as to cause the piezoelectric element array 14 to alternately perform transmission and reception of ultrasonic waves for setting (resetting/updating) a sound velocity (optimal sound velocity) and transmission and reception of ultrasonic waves for producing an ultrasound image and measuring tissue elasticity at predetermined intervals.

In the following explanation, the transmission and reception of ultrasonic waves for setting a sound velocity is also referred to as transmission/reception for sound velocity setting, for convenience. Further, in the following explanation, the transmission and reception of ultrasonic waves for producing an ultrasound image and measuring the tissue elasticity is also referred to as transmission/reception for output, for convenience.

The measurement of tissue elasticity is performed by compressing the subject by the probe 12.

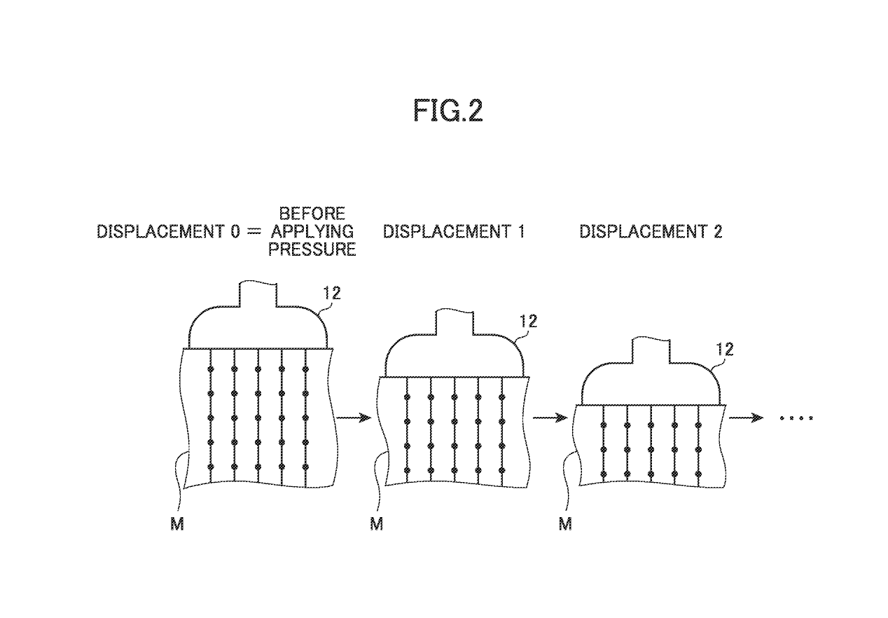

Specifically, in the elasticity measurement mode, the operator (doctor) compresses a subject M with gradually increasing force with the use of the probe 12 according to, for instance, the display on the display unit 30, as conceptually shown in FIG. 2.

In this example, in the transmission/reception for output, transmission and reception having a predetermined focal point is performed one time for one sound ray signal to be produced (for a certain position in the azimuth direction). Alternatively, in this example, in the transmission/reception for output, transmission and reception is performed two times with different focal points (focal points different in position in the depth direction) for one sound ray signal to be produced.

On the other hand, in the transmission/reception for sound velocity setting in the ultrasound diagnostic apparatus 10, transmission and reception of ultrasonic waves is performed more times than the number of times the transmission/reception for output is performed with mutually different focal points for one sound ray signal to be produced. In addition, the number of sound ray signals (the density of sound rays in the azimuth direction) may also be increased more than that in the transmission/reception for output.

It should be noted that, in the present invention, the number of focal points corresponding to one sound ray signal or the number of sound ray signals may differ between transmission and reception of ultrasonic waves for producing an ultrasound image and measuring tissue elasticity (transmission/reception for output) and transmission and reception of ultrasonic waves only for producing an ordinary ultrasound image with no measurement of tissue elasticity.

FIG. 2 also conceptually illustrates an example of the transmission/reception for sound velocity setting.

In FIG. 2, the solid lines in the subject M represent sound ray signals to be produced (i.e., scanning lines to be produced). Points on the sound ray signals represent focal points of transmitted ultrasonic beams. Specifically, in this example of the transmission/reception for sound velocity setting, transmission and reception of ultrasonic beams is performed five times with mutually different focal points to produce one sound ray signal.

Reception signals output from the respective piezoelectric elements of the piezoelectric element array 14 in response to the above-described transmission/reception for sound velocity setting undergo amplification and A/D conversion by the reception circuit 18, and the resulting pieces of reception data are sequentially stored in the reception data memory 36.

At the same time, the sound velocity setting unit 40 supplies a first set sound velocity S1 to the signal processor 20.

The signal processor 20 reads out the pieces of reception data stored in the reception data memory 36, implements delay correction on the pieces of reception data based on the supplied first set sound velocity S1 to produce pieces of delay data, and adds the produced pieces of delay data to perform the reception focusing process to thereby produce a sound ray signal. The signal processor 20 further implements the correction of attenuation and the envelope detection process on the sound ray signal to thereby produce a B-mode image signal.

The B-mode image signal undergoes raster conversion by the DSC 24 and then various kinds of image processing by the image processor 26, and subsequently, is stored in the image memory 32 as a B-mode image signal for sound velocity setting.

The sound velocity setting unit 40 provides a plurality of set sound velocities S1 to Sn to the signal processor 20 in sequence, and B-mode image signals corresponding to those set sound velocities S1 to Sn are produced by the ultrasound image producer 50 and stored in the image memory 32.

After the B-mode image signals corresponding to the set sound velocities S1 to Sn are stored in the image memory 32, the sound velocity setting unit 40 performs the analysis on the B-mode image signals stored in the image memory 32. Based on the results of the analysis of the B-mode image signals, the sound velocity setting unit 40 sets a sound velocity at which the contrast or the sharpness of the image is highest as the optimal sound velocity of the subject of that moment (e.g., under the condition of Displacement 0, Displacement 1 or Displacement 2 shown in FIG. 2).

In setting optimal sound velocities, the analysis of the B-mode image signals is performed for each of segment regions obtained by dividing the subject (ultrasound image), and an optimal sound velocity is set for each of the segment regions. Specifically, a sound velocity at which the contrast or the sharpness of the image is highest is selected to be set as the optimal sound velocity for each of the segment regions.

In the illustrated example, as an example, the subject is divided in the azimuth direction and in the direction parallel to a sound ray to establish a grid pattern with focal points of ultrasonic beams being taken as centers of the respective segment regions, and an optimal sound velocity is set for each of the thus-obtained segment regions. Specifically, an optimal sound velocity is set to correspond to each focal point that is formed in the transmission/reception for sound velocity setting.

Thus, the optimal sound velocity is a sound velocity from a segment region to the piezoelectric elements, where the sound velocity is considered as uniform in the subject from the segment region to the piezoelectric elements. In other words, the optimal sound velocity is an average sound velocity in the subject from a segment region to the piezoelectric elements.

It should be noted that division of the subject for which optimal sound velocities are set, i.e., focal points formed in the transmission/reception for sound velocity setting, may be suitably set in accordance with, for instance, required accuracy of tissue elasticity measurement, required image quality, or required processing speed.

Preferably, focal points are each formed at the same position in every pixel of an ultrasound image to be produced. Alternatively, one focal point may be given for several pixels whose number is appropriately determined in such a manner of giving, for example, one focal point per three pixels, nine pixels, and so forth. Still alternatively, segment regions may be set by equally dividing an ultrasound image by an appropriately-set number, for example, by 10 or 20.

Furthermore, the number of segment regions, the number of focal points on one scanning line, or the like may be determined by the operator. The foregoing setting of segment regions may be made through the operation of mode selection or the like.

A method of setting a sound velocity of a subject is not limited to the foregoing method and use may be made of various known sound velocity setting methods employed in ultrasound diagnostic apparatuses or ultrasound image generating methods.

In the ultrasound diagnostic apparatus 10, setting (resetting/updating) of sound velocities is performed at appropriately-set predetermined timing, in addition to the time of measuring tissue elasticity.

Various kinds of timing can be applied for setting optimal sound velocities. For example, an optimal sound velocity may be set at the start of a diagnosis, set for every predetermined number of frames, set when the probe 12 has been moved by a predetermined distance or more, or set when the probe 12 has remained at one place for a predetermined period of time or more.

The optimal sound velocities of the respective segment regions set by the sound velocity setting unit 40 are each linked with a relevant segment region, supplied to the signal processor 20, and stored therein. Alternatively, the optimal sound velocities may be linked with the relevant segment regions, supplied to the storage unit 48, and stored therein, so that the controller 42 reads out the optimal sound velocities from the storage unit 48 to supply them to the signal processor 20.

As described above, in the elasticity measurement mode, the transmission/reception for sound velocity setting and the transmission/reception for output are alternately performed at predetermined intervals.

After the transmission/reception for sound velocity setting, the transmission/reception for output is performed so that the piezoelectric element array 14 outputs a reception signal resulting from the transmission/reception for output. The reception signal is similarly processed by the reception circuit 18 and the resulting reception data is supplied to the signal processor 20. Alternatively, as necessary, the reception data may be stored in the reception data memory 36, and the signal processor 20 may read out the reception data from the reception data memory 36 and subject the reception data to processing below.

The signal processor 20 having acquired the reception data performs dedicated delay correction on each piece of the reception data based on the stored optimal sound velocity of each segment region as set in the latest transmission/reception for sound velocity setting (i.e., the updated optimal sound velocity of each segment region), thereby producing pieces of delay correction data.

Subsequently, the signal processor 20 adds the produced pieces of delay correction data to perform the reception focusing process to thereby produce a sound ray signal.

The signal processor 20 supplies the produced sound ray signal to the elasticity calculator 34. In addition, the signal processor 20 performs the correction of attenuation and the envelope detection process on the sound ray signal to produce a B-mode image signal.

The B-mode image signal produced by the signal processor 20 undergoes raster conversion by the DSC 24 and predetermined image processing by the image processor 26, whereby a B-mode image signal for use in display is produced.

At this time, in the elasticity measurement mode, the transmission/reception for sound velocity setting and the transmission/reception for output are alternately performed at predetermined intervals, as described above. Accordingly, the ultrasound image producer 50 produces a sound ray signal based on the optimal sound velocity updated in the latest transmission/reception for sound velocity setting, to thereby produce a B-mode image signal.

Further, in the elasticity measurement mode, the operator presses the subject M with gradually increasing force with the use of the probe 12 in accordance with, for instance, the display on the display unit 30, as conceptually shown in FIG. 2.

The sound ray signal produced through the transmission/reception for output based on the optimal sound velocity set in the latest transmission/reception for sound velocity setting is supplied to the elasticity calculator 34.

The elasticity calculator 34 calculates tissue elasticity of the subject with respect to sound ray signals produced through two times of the transmission/reception for output as consecutively carried out by using the correlation of pixels on the sound ray signals (the correlation at the same region on the sound ray signals).

As described above, a known method for tissue elasticity calculation in which the correlation between ultrasound images (sound ray signals) produced with a different degree of pressure is obtained to calculate tissue elasticity can be used.

For instance, the elasticity calculator 34 calculates tissue elasticity under the state of Displacement 1 in FIG. 2 based on the correlation between a sound ray signal produced through the transmission/reception for output in the state of Displacement 0 and another sound ray signal produced through the subsequent transmission/reception for output in the state of Displacement 1.

The elasticity calculator 34 then calculates tissue elasticity under the state of Displacement 2 based on the correlation between the sound ray signal in the state of Displacement 1 and a sound ray signal produced through the subsequent transmission/reception for output in the state of Displacement 2.

In this way, the elasticity calculator 34 obtains the correlation between a sound ray signal produced through certain transmission/reception for output and a sound ray signal produced through the subsequent transmission/reception for output to thereby calculate tissue elasticity of the subject under the state in which the subsequent transmission/reception for output is performed.

It should be noted that the calculation method of tissue elasticity is not limited to the one using sound ray signals produced through two times of transmission/reception for output as consecutively carried out. Specifically, in the tissue elasticity calculation, sound ray signals (ultrasound images) produced through the transmission/reception for output carried out at appropriately-set predetermined intervals may be used to calculate tissue elasticity.

The elasticity calculator 34 links the calculation result, i.e., the measurement result of tissue elasticity with a corresponding sound ray signal and sends the measurement result to at least one of the image memory 32, the image processor 26, and the display controller 28.

Upon receiving the measurement result of tissue elasticity, the image memory 32 links the measurement result of tissue elasticity with a B-mode image signal with which the tissue elasticity was measured, and stores the measurement result.

As described above, the image processor 26 produces a B-mode image for use in display through the transmission/reception for output. Upon receiving the measurement result of tissue elasticity, the image processor 26 further produces at least one of an image indicative of the measurement result of tissue elasticity for use in display and an image in which the measurement result of tissue elasticity overlaps its corresponding B-mode image.

The measurement result of tissue elasticity is displayed in a color scale, for instance.

The display controller 28 causes the display unit 30 to display the color scale indicative of the tissue elasticity and the images supplied from the image processor 26 such as the B-mode image and the image of the measurement result of tissue elasticity.

The tissue elasticity measurement by the ultrasound diagnostic apparatus is performed by pressing the subject with the probe 12. At this time, a sound velocity in the subject varies depending on the state of applied pressure. Specifically, a sound velocity in the subject varies in response to pressure applied with the probe 12 during the measurement of tissue elasticity. Consequently, in the tissue elasticity measurement by conventional ultrasound diagnostic apparatuses, a distorted sound ray signal is produced based on an inappropriate sound velocity, so that the measurement result of tissue elasticity is adversely affected by the distortion.

In the present invention, a sound velocity is updated (reset), the updated sound velocity is used to produce a sound ray signal (ultrasound image), and the sound ray signal produced based on the appropriate sound velocity is used to measure tissue elasticity. Therefore, according to the present invention, tissue elasticity of the subject can be accurately measured based on the appropriate sound velocity.

In the present invention, while it is preferred that, along with the measurement result of tissue elasticity, its corresponding B-mode image (ultrasound image) be also produced, as a further preferred alternative embodiment, this B-mode image is produced based on the updated sound velocity as well. Accordingly, this embodiment makes it possible to carry out a further accurate diagnosis by observing the B-mode image produced based on the appropriate sound velocity along with the accurate tissue elasticity.

The display of a measurement result of tissue elasticity in the display unit 30 may only have the measurement result of tissue elasticity. Preferably, however, the display unit 30 displays the measurement result of tissue elasticity along with a B-mode image.

A measurement result of tissue elasticity and a B-mode image may be displayed in various arrangement styles. For instance, a measurement result of tissue elasticity and its corresponding B-mode image may be arranged side by side. A measurement result of tissue elasticity may overlap its corresponding B-mode image in display. An image in which a measurement result of tissue elasticity overlaps its corresponding B-mode image, and this corresponding B-mode image may be arranged side by side. A measurement result of tissue elasticity and an image in which the measurement result of tissue elasticity overlaps its corresponding B-mode image may be arranged side by side. Any two or more of the foregoing arrangement styles may be simultaneously applied.

Meanwhile, in the elasticity measurement mode, a sound velocity in the subject is considered to hardly vary under the condition where there is no change in the pressure applied to the subject through the probe 12, i.e., the probe 12 is not displaced. Accordingly, updating the sound velocity under this condition leads to useless calculation and also induces the decrease in the frame rate.

To cope with it, in the ultrasound diagnostic apparatus 10, displacement detection means (displacement detector) may be provided at the probe 12 to control the transmission and the reception of ultrasonic waves in accordance with the displacement of the probe 12 in the elasticity measurement mode. Specifically, the displacement of the probe 12, i.e., the piezoelectric element array 14 is detected in the elasticity measurement mode. Under the condition where there is no displacement of the piezoelectric element array 14, which is determined based on the aforesaid displacement detection, only the transmission/reception for output may be performed at predetermined intervals with no transmission/reception for sound velocity setting, and based on a sound velocity acquired in the latest update, the above-described calculation of tissue elasticity of the subject and possibly the production of a B-mode image may be performed.

As the displacement detection means of the piezoelectric element array 14, one exemplary method is, for instance, providing a pressure gauge at the probe 12 to detect pressure applied to the probe 12, and based thereon, detect the displacement of the piezoelectric element array 14. Alternatively, a method in which a displacement sensor such as an acceleration sensor is provided at the probe 12 and based on a result of the measurement by the displacement sensor, the displacement of the piezoelectric element array 14 is detected, may be employed. Still alternatively, use may be made of a method in which a sound ray signal produced by the signal processor 20 or a B-mode image produced by the image processor 26 is analyzed and based on a variation in the image or the like, the displacement of the piezoelectric element array 14 is detected.

In the foregoing example, in the elasticity measurement mode, the transmission/reception for sound velocity setting and the transmission/reception for output are alternately performed, so that a sound velocity is updated by using a reception signal obtained through the transmission/reception for sound velocity setting while the measurement of tissue elasticity and the production of a B-mode image are performed by using a reception signal obtained through the transmission/reception for output.

However, the present invention is not limited thereto. In the elasticity measurement mode, the update of a sound velocity, the measurement of tissue elasticity of a subject and the production of a B-mode image may be carried out by performing only the transmission/reception for sound velocity setting and using a reception signal obtained through the transmission/reception for sound velocity setting.

Specifically, in this alternative embodiment, when the ultrasound diagnostic apparatus 10 is in the elasticity measurement mode, the piezoelectric element array 14 performs only the transmission/reception for sound velocity setting at predetermined intervals.

A reception signal obtained through this transmission and reception is processed by the reception circuit 18, output as reception data, and stored in the reception data memory 36. When the reception data is stored in the reception data memory 36, the signal processor 20 reads out the reception data and the sound velocity setting unit 40 updates the sound velocity, similarly to the above-described configuration. When the sound velocity is updated and supplied, the signal processor 20 reads out the reception data from the reception data memory 36 and produces a sound ray signal with the updated sound velocity. After the sound ray signal is produced, the elasticity calculator 34 calculates tissue elasticity while the ultrasound image producer 50 produces a B-mode image, and the calculation result and the B-mode image are displayed on the display unit 30 in the same manner as the above-described configuration.

Alternatively, in order to improve the frame rate, only the production of a B-mode image may be performed not based on a sound velocity most recently updated but based on a sound velocity before the most-recently-updated sound velocity, although this method is disadvantageous in terms of the image quality.

Still alternatively, in the present invention, the transmission and reception of ultrasonic waves for measuring tissue elasticity and the transmission and reception of ultrasonic waves for producing an ultrasound image corresponding to the elasticity measurement may be separately performed. In this case, preferably, the ultrasound image is produced based on a sound velocity most recently updated.

While the ultrasound diagnostic apparatus, the tissue elasticity measurement method, and the recording medium according to the invention have been described above in detail, the invention is by no means limited to the above embodiments, and various improvements and modifications may be made without departing from the scope and spirit of the invention.

The ultrasound probe can be advantageously employed in an ultrasound diagnostic apparatus used for various diagnoses in the medical settings.

What is claimed is:

1. An ultrasound diagnostic apparatus comprising:
a piezoelectric element array having piezoelectric elements arranged therein, each adapted to transmit ultrasonic waves, receive an ultrasonic echo reflected by a subject, and output a reception signal according to received ultrasonic waves;
a controller adapted to control transmission and reception of ultrasonic waves by the piezoelectric element array;
a storage unit adapted to store the reception signal output by the piezoelectric element array;
a sound velocity setting unit adapted to divide the subject into multiple segment regions and set a sound velocity for each of the segment regions with use of the reception signal stored in the storage unit;
an image producer adapted to produce an ultrasound image by processing the reception signal output by the piezoelectric element array or the reception signal read out from the storage unit based on the sound velocity for each of the segment regions;
a tissue elasticity calculator adapted to calculate tissue elasticity of the subject with use of the reception signal processed by the image producer; and
an image display unit,
wherein when the tissue elasticity calculator calculates the tissue elasticity,
the controller causes the piezoelectric element array to perform transmission and reception of ultrasonic waves for sound velocity setting so that the sound velocity setting unit sets a sound velocity,
the sound velocity setting unit sets the sound velocity for each of the segment regions of the subject with use of the reception signal acquired through the transmission and reception of ultrasonic waves for sound velocity setting, and
the tissue elasticity calculator calculates the tissue elasticity of the subject with use of the reception signal processed by the image producer based on the sound velocity set through the transmission and reception of ultrasonic waves for sound velocity setting.

2. The ultrasound diagnostic apparatus according to claim 1, wherein the image producer produces the ultrasound image by processing the reception signal output by the piezoelectric element array based on the sound velocity set through the transmission and reception of ultrasonic waves for sound velocity setting when the tissue elasticity calculator calculates the tissue elasticity.

3. The ultrasound diagnostic apparatus according to claim 1, wherein a calculation result of the tissue elasticity calculated by the tissue elasticity calculator and the ultrasound image produced by the image producer are displayed on the image display unit.

4. The ultrasound diagnostic apparatus according to claim 3, wherein the image display unit performs display in at least one of an arrangement having an image in which the calculation result of the tissue elasticity overlaps the ultrasound image; an arrangement having the calculation result of the tissue elasticity and the ultrasound image arranged side by side; an arrangement having the image in which the calculation result of the tissue elasticity overlaps the ultrasound image and the ultrasound image arranged side by side; and an arrangement having the image in which the calculation result of the tissue elasticity overlaps the ultrasound image and the calculation result of the tissue elasticity arranged side by side.

5. The ultrasound diagnostic apparatus according to claim 1, further comprising a displacement detector adapted to detect displacement of the piezoelectric element array,
wherein when the tissue elasticity calculator calculates the tissue elasticity of the subject, in a case where the displacement detector detects that there is no displacement of the piezoelectric element array, the controller does not allow the piezoelectric element array to perform the transmission and reception of ultrasonic waves for sound velocity setting, and the tissue elasticity calculator and the image producer process the reception signal based on a sound velocity of the subject most recently set by the sound velocity setting unit.

6. The ultrasound diagnostic apparatus according to claim 5, wherein the displacement detector is at least one of a pressure gauge provided at an ultrasound probe of the ultrasound diagnostic apparatus, a displacement sensor adapted to detect displacement of the piezoelectric element array, and an image analyzer adapted to analyze the reception signal processed by the image producer.

7. The ultrasound diagnostic apparatus according to claim 1, wherein the tissue elasticity calculator calculates the tissue elasticity of the subject with use of the reception signal acquired through the transmission and reception of ultrasonic waves for sound velocity setting or with use of the reception signal acquired through subsequent transmission and reception of ultrasonic waves as carried out immediately after the transmission and reception of ultrasonic waves for sound velocity setting.

8. The ultrasound diagnostic apparatus according to claim 1, wherein the image producer produces the ultrasound image with use of the reception signal acquired through the transmission and reception of ultrasonic waves for sound velocity setting or with use of the reception signal acquired through subsequent transmission and reception of ultrasonic waves as carried out immediately after the transmission and reception of ultrasonic waves for sound velocity setting when the tissue elasticity calculator calculates the tissue elasticity of the subject.

9. A tissue elasticity measurement method, comprising the steps of:
at a time when tissue elasticity of a subject is measured by an ultrasound diagnostic apparatus,
causing a piezoelectric element array having piezoelectric elements arranged therein, each adapted to transmit ultrasonic waves, receive an ultrasonic echo reflected by the subject, and output a reception signal according to received ultrasonic waves, to perform transmission and reception of ultrasonic waves for sound velocity setting for setting a sound velocity of the subject;
setting the sound velocity for each of segment regions established by dividing the subject based on the reception signal acquired through the transmission and reception of ultrasonic waves for sound velocity setting; and
calculating the tissue elasticity of the subject with use of the reception signal which has been output from the piezoelectric element array and processed based on the sound velocity set through the transmission and reception of ultrasonic waves for sound velocity setting.

10. The tissue elasticity measurement method according to claim 9, further comprising the step of:
producing an ultrasound image by processing the reception signal output by the piezoelectric element array based on the sound velocity set through the transmission and reception of ultrasonic waves for sound velocity setting.

11. A recording medium having stored therein a program that causes a computer to implement:
a step of causing a piezoelectric element array having piezoelectric elements arranged therein, each adapted to transmit ultrasonic waves, receive an ultrasonic echo reflected by a subject, and output a reception signal according to received ultrasonic waves, to perform transmission and reception of ultrasonic waves for sound velocity setting for setting a sound velocity of the subject;
a step of setting the sound velocity for each of segment regions established by dividing the subject based on the reception signal acquired through the transmission and reception of ultrasonic waves for sound velocity setting; and
a step of calculating the tissue elasticity of the subject with use of the reception signal which has been output from the piezoelectric element array and processed based on the sound velocity set through the transmission and reception of ultrasonic waves for sound velocity setting.

12. The recording medium according to claim 11, wherein the stored program causes the computer to further implement a step of producing an ultrasound image by processing the reception signal output by the piezoelectric element array based on the sound velocity set through the transmission and reception of ultrasonic waves for sound velocity setting.
